(19) United States
(12) Patent Application Publication
(10) Pub. No.: US 2026/0262941 A1
(43) Pub. Date: Sep. 10, 2026

(54) MANAGEMENT SYSTEM AND SENSOR

(71) Applicant: NITTO DENKO CORPORATION, Ibaraki-shi, Osaka (JP)

(72) Inventors: Ryotaro IWAMOTO, Ibaraki-shi, Osaka (JP); Shingo MAEDA, Ibaraki-shi, Osaka (JP); Nobuyuki KOZONOI, Ibaraki-shi, Osaka (JP)

(21) Appl. No.: 19/167,272

(22) PCT Filed: Mar. 12, 2024
(86) PCT No.: PCT/JP2024/009455

§ 371 Date: Sep. 19, 2025

(30) Foreign Application Priority Data
Mar. 24, 2023 (JP) 2023-047923

(51) Int. Cl. A61B 5/00; G06T 7/00; G06V 40/10

(52) U.S. Cl. CPC A61B 5/0077; A61B 5/4833; G06T 7/0012; G06V 40/10; G06T 2207/30004; G06V 2201/03

(57) ABSTRACT

A management system includes a device configured to be attached to a human body and a management device, wherein the management device includes an acquisition unit configured to acquire image data including an image of the device attached to the human body and an image indicating a physical characteristic of the human body, an extraction unit configured to extract, from the image data, authentication information identifying the human body and information acquired from the device, and a storage control unit configured to store the information acquired from the device in association with the authentication information in a storage unit.

### TECHNICAL FIELD

The disclosures herein relate to sensors for acquiring biometric information, and management systems for managing biometric information acquired by the sensors using authentication systems.

### BACKGROUND ART

In recent years, technologies have been known for preventing misidentification of measurement data by performing personal authentication based on physical characteristics of a subject and subsequently acquiring measurement data by measuring the physical condition of the authenticated subject.

### CITATION LIST

### Patent Literature

Patent Literature 1: Japanese Laid-Open Patent Publication No. 2005-245956

### SUMMARY OF THE INVENTION

### Problem to be Solved by the Invention

In a related technology described above, if the subject is replaced with a third party after being authenticated based on physical characteristics, the physical condition of the third party may be measured instead. In such a case, there is a possibility of misidentification between information of the subject and that of the third party.

The present disclosure aims to prevent misidentification of information.

### Means of Solving the Problem

A management system includes a device configured to be attached to a human body and a management device, wherein the management device includes an acquisition unit configured to acquire image data including an image of the device attached to the human body and an image indicating a physical characteristic of the human body, an extraction unit configured to extract, from the image data, authentication information identifying the human body and information acquired from the device, and a storage control unit configured to store the information acquired from the device in association with the authentication information in a storage unit.

### Effect of the Invention

Misidentification of information can be prevented.

### BRIEF DESCRIPTION OF THE DRAWINGS

FIG. 1 is a drawing illustrating an example of a system configuration of a management system.

FIG. 2 is a drawing illustrating an example of a hardware configuration of the management device.

FIG. 3 is a drawing illustrating an example of a user database.

FIG. 4 is a drawing illustrating functions of each device in the management system.

FIG. 5 is a sequence diagram illustrating operation of the management system.

FIG. 6 is a drawing illustrating a system configuration of a management system of a modified example.

### DESCRIPTION OF THE PREFERRED EMBODIMENTS

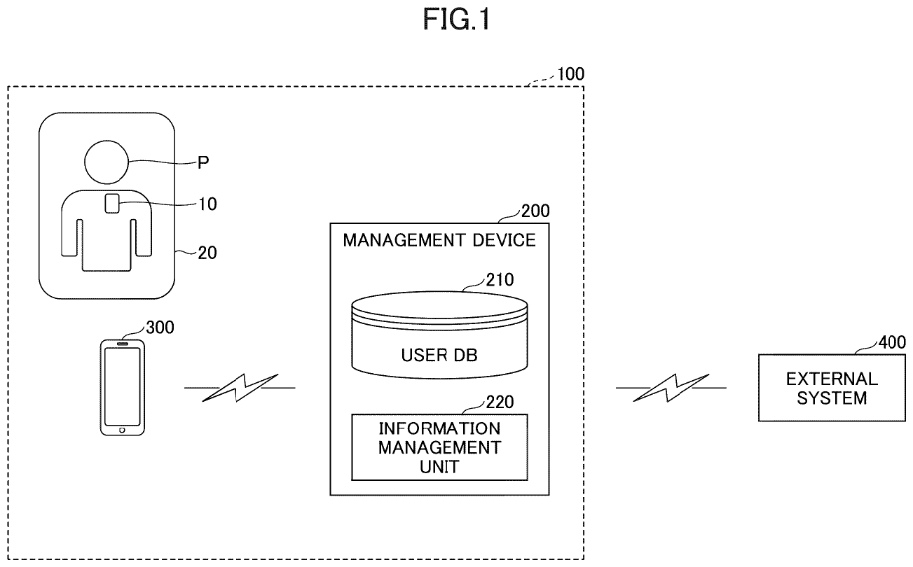

In the following, embodiments of the present invention will be described with reference to the accompanying drawings. FIG. 1 is a drawing illustrating an example of a system configuration of a management system.

A management system 100 of the present embodiment includes a management device 200, a terminal device 300, and a device 10, and the management device 200 and the terminal device 300 are connected via a network. The management device 200 of the present embodiment is connected via a network to an external system 400 existing outside the management system 100.

The external system 400 of the present embodiment is, for example, a system that provides a service using information acquired from the device 10. Details of the external system 400 will be described later.

In the management system 100, the device 10 is configured to be attached to a human body. The device 10 of the present embodiment changes its appearance according to condition of the human body, for example, after being attached to the human body. That is, the appearance of the device 10 attached to the human body can be information indicating a state of the human body. The information indicating the state of the human body can be considered information acquired from the device 10.

In the management system 100 of the present embodiment, the terminal device 300 captures an image 20 of a user P in the state where the device 10 is attached to the body, and transmits image data indicating the image 20 to the management device 200. The terminal device 300 of the present embodiment may be, for example, a smartphone or the like owned by the user P, and has an imaging function and a communication function. The terminal device 300 may be a portable terminal device having an imaging function, or a tablet type terminal or the like.

The image 20 is an image including an image of the device 10 attached to the body of the user P and an image indicating a physical characteristic of the user P. The image indicating the physical characteristic of the user P may be, for example, a face image of the user P. In the present embodiment, the image indicating the physical characteristic of the user P included in the image 20 is used as user authentication information. In the present embodiment, an image of such as an iris, blood vessels in an eyeball, or an ear may be used as the image indicating the physical characteristic of the user P.

In the present embodiment, images of the device 10 whose appearance changes according to the physical condition of the user P are used as information showing the state of the human body of the user P. In the following description, information showing the state of the human body is referred to as biometric information.

The management device 200 of the present embodiment has a user database 210 and an information management unit 220. When the management device 200 of the present embodiment receives image data showing the image 20 from the terminal device 300, the information management unit 220 extracts the authentication information of the user P and the biometric information acquired by the device 10 from the image data. The information management unit 220 stores an analysis result of analyzing the extracted biometric information in association with the authentication information in the user database 210.

When the user P receives service from the external system 400, the management device 200 of the present embodiment transmits the analysis result of the biometric information of the user P stored in the user database 210 to the external system 400. At this time, the management device 200 may transmit the authentication information of the user P to the external system 400 together with the analysis result of the biometric information.

In the present embodiment, since the authentication information and the biometric information of the user P are extracted from a single image as described above, misidentification between the information of a third party and the information of the user P can be prevented. Therefore, according to the present embodiment, the biometric information of the third party is not acquired as the biometric information of the user P, and highly reliable information can be provided to the external system 400.

Moreover, in the present embodiment, by simply attaching the device 10 to the user P and capturing an image of the user P, the analysis results of both authentication information and biometric information can be acquired from a single image. In addition, it is possible to easily acquire biometric information for which authenticity of acquisition from the user P has been verified.

In the management system 100, the management device 200 may be implemented by a plurality of information processing devices. In other words, the information management unit 220 may be a function implemented by a plurality of information processing devices. In the example of FIG. 1, the management device 200 is assumed to have the user database 210, but this is not limited thereto. The user database 210 may be provided in an external device capable of communicating with the management device 200.

Furthermore, in the present embodiment, the terminal device 300 may also serve as the management device 200. That is, the terminal device 300 may have the user database 210 and the information management unit 220.

In this case, the analysis results of authentication information and biometric information of the user P stored in the user database 210 may be transmitted from the terminal device 300 to the external system 400.

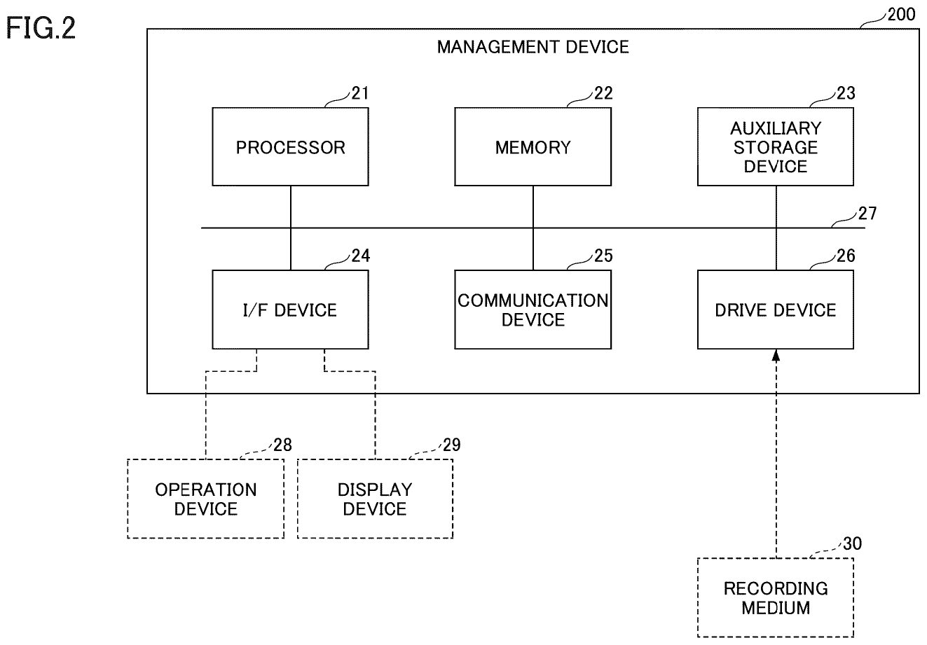

Next, the management device 200 of the present embodiment will be described with reference to FIG. 2. FIG. 2 is a drawing illustrating an example of a hardware configuration of the management device.

The management device 200 of the present embodiment includes a processor 21, a memory 22, an auxiliary storage device 23, an interface (I/F) device 24, a communication device 25, and a drive device 26. The respective hardware components of the management device 200 are connected to each other via a bus 27.

The processor 21 includes various arithmetic devices such as a CPU (Central Processing Unit) and a GPU (Graphics Processing Unit). The processor 21 reads and executes various programs (e.g., information management program) on the memory 22.

The memory 22 includes main storage devices such as ROM (Read Only Memory) and RAM (Random Access Memory). The processor 21 and the memory 22 form what is called a computer, and the computer implements, for example, the information management unit 220 by executing various programs read on the memory 22 by the processor 21.

The auxiliary storage device 23 stores various programs and various data used when the various programs are executed by the processor 21.

The I/F device 24 is a connection device for connecting an operation device 28 and a display device 29, which are examples of external devices, to the management device 200. The I/F device 24 accepts an operation for the management device 200 via the operation device 28. The I/F device 24 may output a processing result of the management device 200, and the result may be displayed to a user of the management device 200 via a display device 29.

The communication device 25 is a communication device for communicating with other devices (in the present embodiment, the terminal device 300 and the external system 400).

The drive device 26 is a device for receiving a recording medium 30. The recording medium 30 here includes a medium for recording information optically, electrically, or magnetically, such as a CD-ROM, a flexible disk, or a magneto-optical disk. The recording medium 30 may also include semiconductor memory for recording information electrically, such as ROM, flash memory, or the like.

Various programs to be installed in the auxiliary storage device 23 may be installed, for example, by inserting a distributed recording medium 30 into the drive device 26 and reading the programs recorded on the recording medium 30 by the drive device 26. Alternatively, various programs to be installed in the auxiliary storage device 23 may be installed by downloading from the network via the communication device 25.

The hardware configuration of the terminal device 300 of the present embodiment may be the same as that of the management device 200 except that the terminal device 300 includes an imaging device.

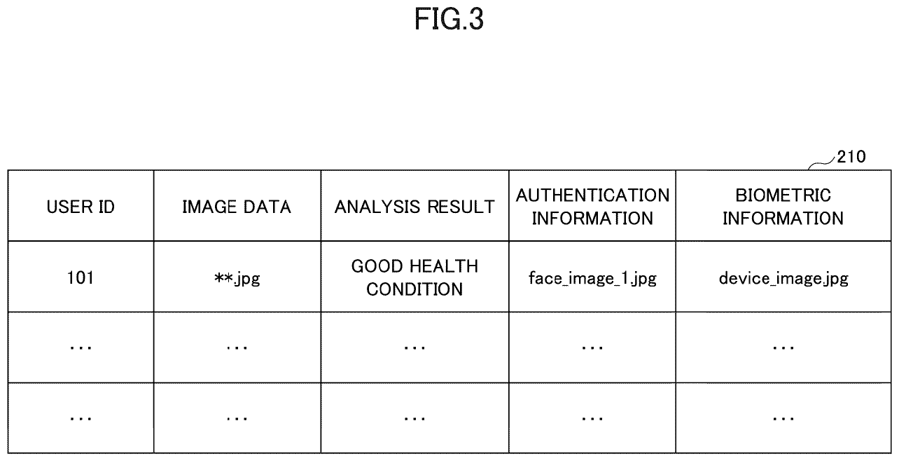

Next, a user database 210 of the present embodiment will be described with reference to FIG. 3. FIG. 3 is a drawing illustrating an example of the user database.

The user database 210 of the present embodiment stores user information related to the user P of the device 10. The user information of the present embodiment includes a user ID, image data, analysis results, authentication information, biometric information and the like as items of information, and an item “user ID” is associated with other items.

A value of the item “user ID” is identification information for specifying a user, and may be identification information common to the external system 400 and the management system 100. The value of the item “image data” is information for specifying image data indicating an image 20 of the user P to which the device 10 is attached. Specifically, the value of the item “image data” may be a file name of image data indicating the image 20. The image data indicating the image 20 may be stored separately in the auxiliary storage device 23.

The value of the item “analysis result” indicates the result of analyzing the image of the device 10 extracted from the image 20. Specifically, the value of the item “analysis result” may be information indicating a health condition of the user P. The image in the present embodiment includes a still image and a moving image. The still image may be an image of one frame cut from a moving image.

The value of the item “authentication information” is information for specifying the face image data of the user P extracted from the image 20. In the present embodiment, the face image data of the user P extracted from the image 20 is stored in the user database 210 separately from the image data indicating the image 20.

The value of the item “biometric information” is information for specifying the image data of the device 10 extracted from the image 20. In the present embodiment, the image data of the device 10 extracted from the image 20 is stored in the user database 210 separately from the image data indicating the image 20.

The information items shown in FIG. 3 are examples, and the information items stored in the user database 210 are not limited to the example shown in FIG. 3. The user database 210 may only store the user ID and the analysis result of biometric information, and does not need to contain any other information items. Further, the user database 210 may include items other than the information items shown in FIG. 3.

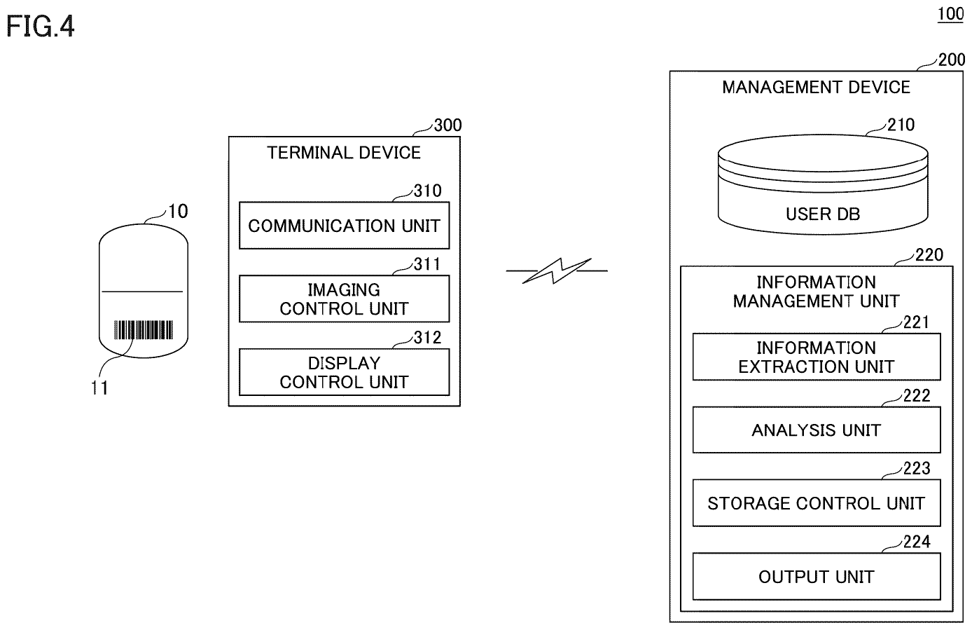

Next, referring to FIG. 4, functions of each device of the management system 100 of the present embodiment will be described. FIG. 4 is a drawing illustrating the functions of each device in the management system.

The terminal device 300 of the present embodiment has a communication unit 310, an imaging control unit 311, and a display control unit 312.

The communication unit 310 controls communication between the terminal device 300 and devices other than the terminal device 300. The imaging control unit 311 controls the imaging function. Specifically, the imaging control unit 311 acquires image data according to the operation of the user P. The display control unit 312 controls various displays on the display device of the terminal device 300.

The management device 200 of the present embodiment has a user database 210 and an information management unit 220. The information management unit 220 includes an information extraction unit 221, an analysis unit 222, a storage control unit 223, and an output unit 224.

The information extraction unit 221 extracts biometric information and authentication information from the image data acquired from the terminal device 300. In other words, the information extraction unit 221 recognizes the face image of the user P included in the image 20 indicated by the image data acquired from the terminal device 300, and extracts the face image data as authentication information from the image data indicated by the image 20.

The information extraction unit 221 recognizes the image of the device 10 included in the image 20 indicated by the image data acquired from the terminal device 300, and extracts the image data of the device 10 as biometric information from the image data indicated by the image 20. That is, the information extraction unit 221 acquires the information from the device 10.

The analysis unit 222 analyzes the body condition of the user P from the biometric information extracted by the information extraction unit 221 and outputs the analysis result.

The storage control unit 223 stores the authentication information the biometric information, and the analysis result extracted by the information extraction unit 221 in association with the user ID in the user database 210. The user ID may be input at the terminal device 300.

The output unit 224 outputs the analysis result of the biometric information of the user P to the external system 400.

The device 10 of the present embodiment will now be described. The device 10 of the present embodiment may be, for example, a colorimetric sensor which, when attached to a human body, changes its color in response to body fluid such as sweat secreted from the human body.

The colorimetric sensor may detect pH of the body fluid of the user P and content of glucose, lactate, sodium, potassium, chloride ion, lactic acid, uric acid, cortisol, and the like in the body fluid.

When the device 10 is a colorimetric sensor, the analysis unit 222 may analyze the health condition of the user P based on the color of the image of the device 10. Specifically, the analysis unit 222 may output presence or absence of diabetes, blood alcohol concentration, stress level, heat stroke level, and the like of the user P as analysis results.

Further, the device 10 of the present embodiment may be a patch containing a drug that is absorbed through skin. In this case, an appearance of the device 10 may change according to the time elapsed since the user P adhered the device 10 to the skin. The appearance of the device 10 in this case may be the color of the device 10, the shape of characters printed on the device 10, and the like.

When the device 10 is a patch, the analysis unit 222 may analyze a state of administration of the drug to the user P from the image of the device 10.

The device 10 of the present embodiment may be a non-invasive sensor. The non-invasive sensor may be, for example, an electric, optical, or temperature sensor. In this case, the device 10 may acquire the body temperature, heart rate, blood pressure, and the like of the user P as biometric information. Like the colorimetric sensor, these non-invasive sensors may detect pH of the body fluid of the user P or the amount of glucose, lactate, sodium, potassium, chloride ion, lactic acid, uric acid, cortisol, or the like in the body fluid. Furthermore, the device 10 may communicate with the terminal device 300 to transmit biometric information to the terminal device 300.

The device 10 of the present embodiment may undergo a further change in appearance when the device is removed from the human body, relative to the appearance the device 10 exhibited while being attached to the body. In other words, in the device 10 of the present embodiment, when the device 10 is re-attached to the user P after once having been attached to the user P, the appearance of the device 10 is different from that before the device 10 was first attached to the user P.

Specifically, the device 10 may have, for example, embedded identification information identifying the device 10 and a two-dimensional code 11 printed thereon. The two-dimensional code 11 of the present embodiment may be of a type that is damaged when the device 10 is removed from the human body after the device 10 is attached to the human body. By providing the two-dimensional code 11 on the device 10, the appearance of the device 10 can be changed when the device 10 is removed from the human body.

Moreover, the device 10 may be configured such that characters or the like appear on the surface (surface opposite to the surface contacting the skin of the human body) of the device 10 when the device 10 is removed from the human body. Moreover, the color of the device 10 may change when the device 10 is removed from the human body.

Note that, the appearance of the device 10 after being removed from the human body may differ from the appearance of the device 10 when attached to the human body, to an extent that the removal of the device 10 is visually recognizable. The manner in which the appearance of the device 10 is changed is not limited to the example described above.

In the present embodiment, the appearance of the device 10 changes when it is peeled off from the human body to which it is attached, so that misidentification of information between the user P and the third party can be prevented.

In other words, in the present embodiment, the appearance of the device 10 is different before versus after it is peeled off from the human body, so that misidentification of the biometric information of the user P and the biometric information of the third party can be prevented, and the authentication information of the user P and the biometric information of the third party are not associated.

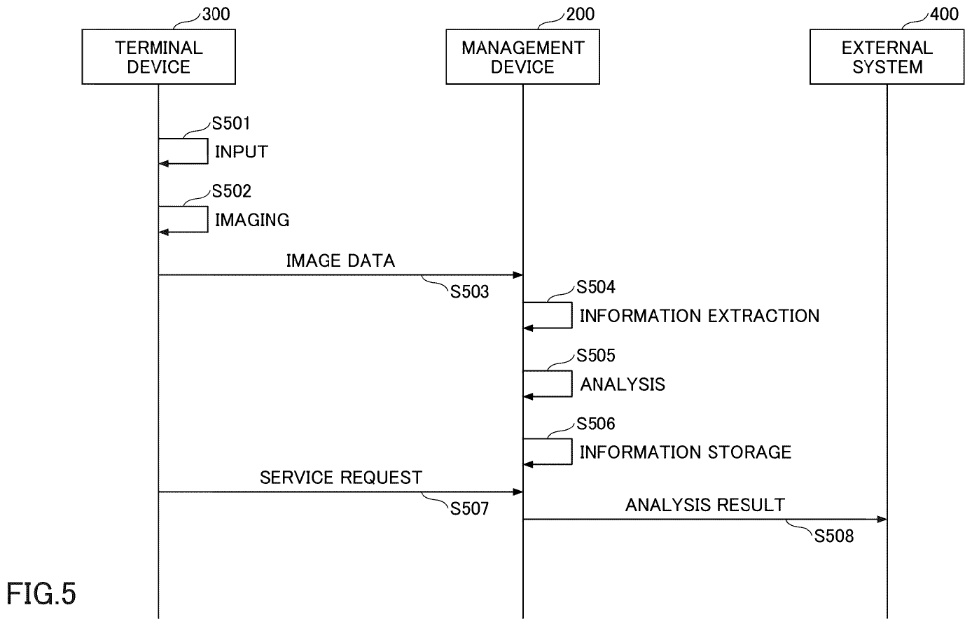

Next, the operation of the management system 100 of the present embodiment will be described with reference to FIG. 5. FIG. 5 is a sequence diagram illustrating operation of the management system.

In the management system 100 of the present embodiment, the terminal device 300 displays, by the display control unit 312, an input screen for prompting the user P to input a user ID, and receives input of the user ID (step S501).

Subsequently, the terminal device 300, in response to an operation by the user P, captures an image of the user P with the device 10 attached controlled by the imaging control unit 311 (step S502), and transmits image data of the captured image 20 to the management device 200 via a communication unit 310 (step S503).

When the management device 200 acquires the image data, the information extraction unit 221 extracts authentication information and biometric information of the user P from the image data (step S504). Subsequently, the management device 200 analyzes the biometric information with the analysis unit 222 (step S505).

Subsequently, the management device 200 stores the authentication information, biometric information, and analysis result of the user P in association with each other in the user database 210 with the storage control unit 223 (step S506).

In the present embodiment, the biometric information and the analysis result stored in the user database 210 are the information confirmed to be the information of the user P.

Subsequently, the management device 200 receives a request for a service provided by the external system 400 from the terminal device 300 (step S507). This request may include, for example, a user ID.

Upon receiving the request, the management device 200 acquires the analysis result of the biometric information associated with the user ID in the user database 210, and outputs it to the external system 400 by the output unit 224 (step S508).

In the present embodiment, by providing the analysis result of the biometric information of the user P to the external system 400 as described above, it is possible to allow the user P to use the service provided by the external system 400.

Here, a use scene of the present embodiment will be described. First, the case where the device 10 is a colorimetric sensor and the external system 400 is a system providing services such as medical insurance will be described.

In this case, the management device 200 extracts the image of the device 10 from the image data of the user P with the device 10 attached, and analyzes the color of the image. Then, the management device 200 may store the result of analyzing the color of the image of the device 10 in the user database 210 as information indicating the health condition of the user.

When the management device 200 receives a request for the external system 400 of the user P, it provides the external system 400 with information indicating the health condition of the user P to the external system 400. The external system 400 may perform various processes using information indicating the health condition of the user P, for example.

In the present embodiment, highly reliable information can be provided to the external system 400 using information indicating the health condition of the user P.

Next, a case where the device 10 is a patch and the external system 400 provides a service for managing the state of administration of the drug of the user P will be described. The drug contained in the device 10 which is a patch may be, for example, a drug for angina pectoris, a drug for asthma, a progesterone preparation, a narcotic analgesic, nicotine, and the like.

When the management device 200 receives image data of the user P with the device 10 attached from the terminal device 300, the time when the image data is received may be stored in the user database 210 in association with the user ID as the time when the administration of the drug to the user P started.

Then, the management device 200 may output to the external system 400 information indicating that the administration of the drug to the user P has started.

Furthermore, the management device 200 may periodically receive image data of the user P with the device 10 attached from the terminal device 300 after the user P has attached the device 10, and analyze image data indicating an image of the device 10 extracted from the image data.

The device 10, which is a patch, may change its color or cause characters to appear according to the amount of the drug administered to the user P, for example. Moreover, the appearance of the device 10 may change when the administration of the drug to the user P is finished. Specifically, the device 10 may change the color of the device 10 when the administration of the drug to the user P is finished. Thus, when the device 10 is a patch, the appearance of the device 10 may change according to the time elapsed since the device 10 was attached to the user P.

The management device 200 may periodically receive image data after the device 10 is attached to the user P, analyze the image data showing the image of the device 10, acquire the amount of the drug administered to the user P as an analysis result, and store it in the user database 210 in association with the time when the image data is received. Then, the management device 200 may output the analysis result to the external system 400 with information showing the state of administration of the drug to the user P.

In the present embodiment, it is possible to provide highly reliable information to the external system 400 managing the state of administration of the drug to the user P.

In the above-described embodiment, the terminal device 300 and the management device 200 are separate devices, but this is not limited thereto.

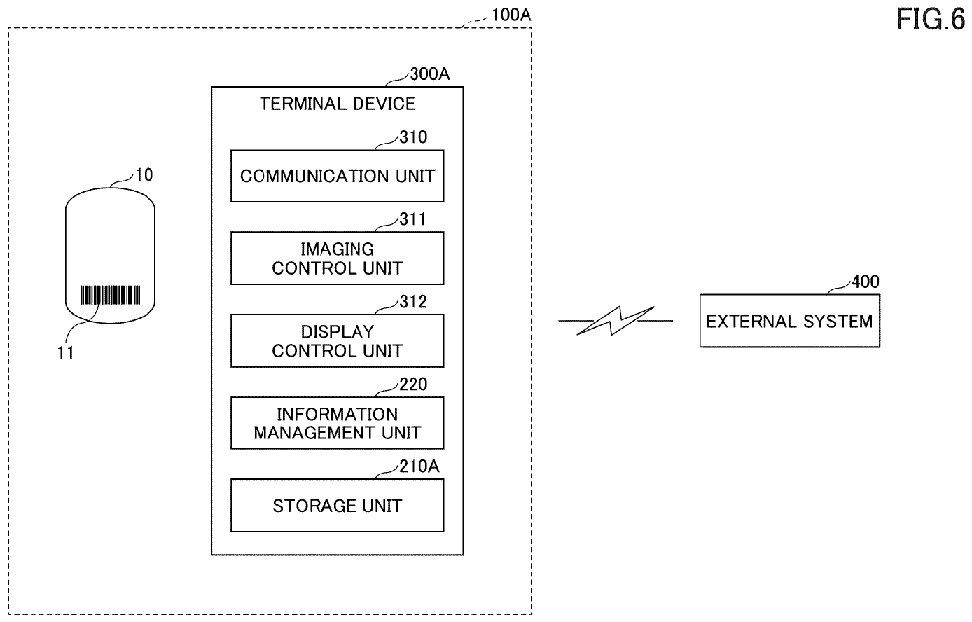

FIG. 6 is a drawing illustrating a system configuration of a management system of a modified example. In a management system 100A shown in FIG. 6, a terminal device 300A also serves as the management device 200. In other words, the terminal device 300A has the information management unit 220 of the management device 200 and a storage unit 210A for storing user information of the user of the terminal device 300.

In FIG. 6, the terminal device 300A acquires image data of the user to which the device 10 is attached by the imaging control unit 311, and associates the analysis result of the biometric information with the authentication information by the information management unit 220 and stores it in the storage unit 210A.

When the terminal device receives a request for the external system 400 from the user, the terminal device 300A outputs to the external system 400 an analysis result of the biometric information stored in the storage unit 210A to the external system 400.

The terminal device 300A shown in FIG. 6 may be configured by downloading a program for implementing the information management unit 220 from a management device (not shown) provided on the Internet to the terminal device 300A.

In the modified example, the terminal device 300A can acquire the authentication information of the user P and the analysis result of the biometric information from one image data, and the management device 200 is not required. Therefore, in the modified example, it can be easily linked with an external system 400 that uses the analysis result of the biometric information.

Further, the present invention is not limited to these embodiments, and various variations and modifications may be made without departing from the scope of the present invention.

The present application is based on and claims priority to Japanese patent application No. 2023-047923 filed on Mar. 24, 2023, with the Japan Patent Office, the entire contents of which are hereby incorporated by reference.

### REFERENCE SIGNS LIST

10 device
100, 100A management system
200 management device
210 user database
220 information management unit
221 information extraction unit
222 analysis unit
223 storage control unit
224 output unit
300, 300A terminal device
400 external system

1. A management system comprising a device configured to be attached to a human body and a management device, wherein the management device includes:
an acquisition unit configured to acquire image data including an image of the device attached to the human body and an image indicating a physical characteristic of the human body;
an extraction unit configured to extract, from the image data, authentication information identifying the human body and information acquired from the device; and
a storage control unit configured to store the information acquired from the device in association with the authentication information in a storage unit.

212. The management system according to claim 1, wherein:
the information acquired from the device is biometric information of the human body;
the image indicating the physical characteristic of the human body is an image including at least one of a face, an iris, blood vessels in an eyeball, or an ear of the human body;
the authentication information is image data indicating the physical characteristic of the human body;
the management device further includes an analysis unit configured to analyze the biometric information of the human body; and
the storage control unit is configured to store an analysis result of the biometric information in association with the authentication information in the storage unit.

3. The management system according to claim 2, wherein the device is at least one of a colorimetric sensor or a non-invasive sensor.

4. The management system according to claim 3, wherein when the device is the colorimetric sensor or the non-invasive sensor:
the biometric information is image data including an image of the colorimetric sensor or the non-invasive sensor; and
the analysis result of the biometric information is information indicating a health condition of the human body.

5. The management system according to claim 2, wherein:
the device is a patch containing a drug;
the biometric information is image data including an image of the patch; and
the analysis result of the biometric information is information indicating a state of administration of the drug to the human body.

6. The management system according to claim 5, wherein the storage control unit is configured to store information indicating time at which the image data including the image of the patch was acquired in association with the authentication information in the storage unit.

7. The management system according to claim 6, wherein an appearance of the device changes, after the administration of the drug to the human body is completed, from an appearance during the administration.

8. The management system according to claim 1, wherein the device is configured to undergo a change in appearance when the device is removed from the human body.

9. A device configured to be attached to a human body, to acquire biological information from the human body, and to undergo a change in appearance when removed from the human body.
